(19) United States
(12) Patent Application Publication
(10) Pub. No.: US 2020/0039922 A1
(43) Pub. Date: Feb. 6, 2020

(54) HIGH MOBILITY GROUP B1 PROTEIN INHIBITORS

(71) Applicant: AFECTA PHARMACEUTICALS, INC., Irvine, CA (US)

(72) Inventors: Bruce KOVACS, Long Beach, CA (US); Kamron SHAHBAHRAMI, Irvine, CA (US); George NICOLA, San Diego, CA (US)

(21) Appl. No.: 16/339,324

(22) PCT Filed: Oct. 4, 2017
(86) PCT No.: PCT/US17/55041

§ 371 Date: Apr. 3, 2019

Related U.S. Application Data
(60) Provisional application No. 62/404,508, filed on Oct. 5, 2016.

(51) Int. Cl. C07C 233/08; C07C 229/38

(52) U.S. Cl. CPC C07C 233/08; C07C 229/38

(57) ABSTRACT

Compounds, pharmaceutically acceptable salts, and pharmaceutical compositions thereof are disclosed that are useful for inhibition of the biological activity of HMGB1, as well as methods of treatment for diseases involving the inflammation activity or cytokine activity of HMGB1.

### FIELD OF THE INVENTION

This invention pertains to inhibitors of High Mobility Group B1 protein (HMGB1). The invention provides novel compounds useful in the treatment of autoimmune, inflammatory degenerative and metabolic diseases associated with HMGB1 and its signaling pathways, such as Toll like receptors 2 and 4 (TLR2 and TLR4) and Receptor for Advanced Glycation End-products (RAGE).

### BACKGROUND OF THE INVENTION

In general, inflammation may be thought of as a complex protective reaction of the immune system and microcirculation, initiated when there is infection, injury, presence of atypical or foreign proteins, nucleic acids, and/or chemicals in cells or tissues. Local and systemic inflammatory responses aim to eliminate the inciting stimulus, promote tissue repair and healing and, in the case of infection, establish immune memory such that the host mounts a faster and more specific response on a future encounter. Acute and chronic inflammatory responses are complex but highly coordinated sequences of events involving molecular, cellular and physiological alterations. These responses often begin with the production of soluble mediators (including complement, chemokines, cytokines, free radicals, vasoactive amrnines and eicosanoids (such as prostaglandins)) by resident cells (that is; tissue macrophages, dendritic cells (DCs), lymphocytes, endothelial cells, fibroblasts and mast cells) in the injured or infected tissue. Concomitantly, cell adhesion molecules are upregulated on circulating leukocytes and endothelial cells, promoting the exudation of proteins and influx of granulocytes from the blood. Upon arrival, these leukocytes (typically polymorphonuclear cells (PMNs) in the case of nonspecific inflammation, or eosinophils in response to allergens) primarily function to phagocytose and eliminate tissue debris and microorganisms through distinct intracellular mechanisms (for example, involving superoxide radicals, myeloperoxidase, proteases and lactoferrins) and/or extracellular mechanisms (such as neutrophil extracellular traps). In some circumstances inflammation can damage normal cells, tissues, and organs. It has been recognized that some diseases that are associated with chronic inflammation are driven by ongoing pro-inflammatory processes, and that therapy based on rectifying these defects will help to guide ongoing inflammation down a pro-resolution pathway. This well-characterized phase of the inflammatory response is routinely targeted using drugs such as non-steroidal anti-inflammatory drugs (NSAIDs) and pro-inflammatory cytokine-negating biologics (for example, tumor necrosis factor (TNF)-specific antibodies) that inhibit or antagonize the action of these mediators. Such drugs currently form the mainstay for the treatment of chronic inflammatory disease.

High-mobility group box 1 protein (HMGB1), also known as High Mobility Group Protein 1 (HMG-1) and amphoterin, is a member of a family of DNA-binding proteins termed High Mobility Group (HMG). HMG family members contain an HMG-box domain which contains three alpha helices separated by loops and bind only non-B-type DNA confirmations (i.e. kinked or unwound DNA) with high affinity. HMGB1 is a non-histone chromosomal protein and has also been shown to be a soluble protein that binds to several membrane bound cytokine receptors and acts as a pro-inflammatory cytokine (1-3). Pro-inflammatory cytokines are to be distinguished from cytokines that have predominately anti-inflammatory activity such as IL-1ra, IL-11, and IL-10, which do not induce or exacerbate the inflammatory process (4).

HMGB1 is expressed at high levels in the nucleus of almost all cells (1). It was originally discovered as a nuclear protein that could bend DNA. It is now known that HMGB1 can also act extracellularly, both as an inflammatory mediator that promotes monocyte migration and cytokine secretion, and as a mediator of T cell-dendritic cell interaction (1-4). The cytokine activity of HMGB1 is restricted to the HMG B box (5). HMGB1 is released in response to cell death as a secretion product into the extracellular milieu. Although HMGB-1 does not possess a classic signal sequence, it appears to be secreted as an acetylated form via secretory endolysosome exocytosis. Once secreted, HMGB1 transduces cellular signals through high affinity receptors such as RAGE, TLR2, and TLR4. TLR2 and TLR4 activate the MyD88-dependent intracellular signaling pathway. HMGB1 signal transduction via the RAGE receptor however, is MyD88 independent (7, 8).

There is a need for new therapeutic agents which suppress the production, release, and/or biological activity of pro-inflammatory cytokines such as, but not limited to: Interleukin 1b (IL-1b), Interleukin 6 (IL-6), Interleukin 17 (IL-17), and Tumor Necrosis Factor alpha (TNF-alpha). These pro-inflammatory cytokines are induced by extracellular HMGB1 through its interaction with TLR2/TLR4 and RAGE receptors in human cells and which therefore can be used in treatment of many disorders associated with extracellular presence of HMGB1.

### SUMMARY OF THE INVENTION

According to one embodiment of the present invention, there is provided a compound of Formula 1 below:

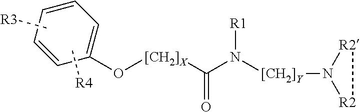

wherein:

R1 is independently selected from methyl, ethyl, propyl, butyl, haloalkyl, or hydrogen;

R2 and R2′ are independently selected from methyl, ethyl, propyl, butyl, alkoxy, haloalkyl, or hydrogen. Alternatively, R2 and R2′ are fused into a heterocyclic ring with 5 or 6 members;

R3 is independently selected from hydroxyl, alkoxy, carboxylic acid, or hydrogen;

R4 is independently selected from hydrogen, hydroxyl, or halogen;

X is 1-3;

Y is 1-3;

or a pharmaceutically acceptable salt, addition compound, or pro-drug thereof.

According to another embodiment of the present invention, there is provided a compound of Formula 2 below:

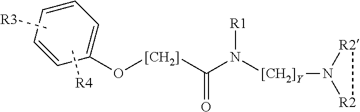

wherein:

R1 is independently selected from methyl, ethyl, or hydrogen;

R2 and R2′ are independently selected from methyl, ethyl, n-propyl, 2-propyl, tert-butyl, alkoxy, or hydrogen;

R3 is independently selected from methyl, ethyl, hydroxyl, carboxylic acid, alkoxy or hydrogen;

R4 is independently selected from hydrogen, hydroxyl or halogen;

Y is 1-3;

or a pharmaceutically acceptable salt, addition compound, or pro-drug thereof.

Additional aspects of the invention include a pharmaceutical composition comprising a compound as defined above and a pharmaceutically acceptable carrier or diluent therefor.

Yet another aspect of the invention is suppression of production, release, and/or biological activity of pro-inflammatory cytokines. Still another aspect of the invention is suppression of at least one inflammatory cytokine cascade mediated by HMGB1.

A further aspect of the invention is the use of a compound as defined above in the manufacture of a medicament for the treatment of a subject at risk for or having at least one inflammatory cytokine cascade disorder associated with increased tissue or blood levels of HMGB1 acting through its interaction with TLR2/TLR4 and RAGE receptors. Examples of such disorders are: autoimmune diseases; inflammatory diseases; auto-inflammatory conditions; pain conditions; respiratory ailments; airway and pulmonary conditions; gastrointestinal disorders; allergic diseases; atopic disorders, infection-based diseases; trauma and tissue injury-based conditions; fibrotic diseases; ophthalmic/ocular diseases; joint, muscle, and bone disorders; skin/dermatological diseases; renal diseases; genetic diseases; hematopoietic diseases; liver diseases; oral diseases; metabolic diseases, including diabetes (e.g. Type II) and complications thereof; proliferative diseases; cardiovascular conditions; vascular conditions; neuro-inflammatory conditions; neurodegenerative conditions; cancer; sepsis; pulmonary inflammation and injury; and pulmonary hypertension.

The invention is also directed to a method of treating a subject at risk for or having a condition mediated by an inflammatory cytokine cascade comprising administering to a subject an amount of a compound of the invention for the treatment of an inflammatory cytokine cascade disease or disorder mediated by HMGB1 through its interaction with TLR2/TLR4 and RAGE receptors. Inflammatory cytokine cascade diseases and disorders mediated by HMGB1 include, but are not limited to: autoimmune diseases; inflammatory diseases; autoinflammatory conditions; pain conditions; respiratory ailments; airway and pulmonary conditions; gastrointestinal disorders; allergic diseases; atopic disorders, infection-based diseases; trauma and tissue injury-based conditions; fibrotic diseases; ophthalmic/ocular diseases; joint, muscle and bone disorders; skin/dermatological diseases; renal diseases; genetic diseases; hematopoietic diseases; liver diseases; oral diseases; metabolic diseases, including diabetes (e.g. Type II) and complications thereof, proliferative diseases; cardiovascular conditions; vascular conditions; neuro-inflammatory conditions; neurodegenerative conditions; cancer; sepsis; pulmonary inflammation and injury; and pulmonary hypertension.

Yet another aspect of the invention is a compound that inhibits the biological pro-inflammatory activity of HMGB1 and decreases the MyD88 dependent and RAGE dependent (MyD88 independent) signaling cascades by simultaneously decreasing the levels of other components of these pathways that occur due to HMGB1 interaction with TLR2/TLR4 and RAGE receptors, such as transcription factors including but not limited to NF-κβ, AP-1, CREB, STAT3, myogenin, and/or Sp1.

### BRIEF DESCRIPTIONS OF THE DRAWINGS

FIG. 1 shows that treatment with compounds of the invention reduces expression of TLR2/TLR4 (MyD88 dependent) and RAGE (MyD88 independent) signaling pathway genes that are upregulated by HMGB1. 1A and 1B: TLR2 gene expression. 1C and 1D: MCP1/CCL2 gene expression.

FIG. 2 shows that treatment with compounds of the invention reduces expression of TLR2/TLR4 (MyD88 dependent) and RAGE (MyD88 independent) signaling pathway genes that are upregulated by HMGB1. 2A and 2B: NF-kB gene expression. 2C and 2D: VCAM gene expression.

FIG. 3 shows that treatment with compounds of the invention reduces HMGB1 induced cytokine release from cells due to TLR2/TLR4 (MyD88 dependent) signaling. 3A and 3B: IL-6 supernate. 3C and 3D: TNF-alpha supernate.

FIG. 4 shows that treatment with compounds of the invention reduces HMGB1 induced cytokine release from cells due to TLR2/TLR4 (MyD88 dependent) signaling. 4A and 4B IL-1b supernate. 4C and 4D: IL-17 supernate.

FIG. 5 shows that treatment with compounds of the invention reduces HMGB1 induced cytokine release from cells by RAGE (MyD88 independent) signaling.

TABLE 1 shows various compounds according to Formula 1 and Formula 2.

### DETAILED DESCRIPTION OF THE INVENTION

### Definitions

“Addition compound” refers to a complex of two or more complete molecules in which each preserves its fundamental structure and no covalent bonds are made or broken (for example, hydrates of salts, adducts).

“Alkyl” refers to a saturated linear monovalent hydrocarbon moiety of one to twelve, typically one to six, carbon atoms or a saturated branched monovalent hydrocarbon moiety of three to twelve, typically three to six, carbon atoms. Exemplary alkyl group include, but are not limited to, methyl, ethyl, n-propyl, 2-propyl, tert-butyl, pentyl, and the like. “Lower alkyl” refers to a straight or branched hydrocarbon containing 1-4 carbon atoms.

“Optionally-substituted alkyl” refers to an alkyl group as defined herein in which one or more hydrogen atom(s) is optionally replaced with a substituent such as halide, hydroxyl, alkoxy, or other heteroatom substituent.

“Alkene” refers to an unsaturated linear divalent hydrocarbon moiety of one to twelve, typically one to six, carbon atoms or a saturated branched divalent hydrocarbon moiety of three to twelve, typically three to six, carbon atoms. Exemplary alkene groups include, but are not limited to, methylene, ethylene, propylene, butylene, pentylene, and the like.

“Alkenyl” refers to a linear monovalent hydrocarbon moiety of two to ten carbon atoms or a branched monovalent hydrocarbon moiety of three to ten carbon atoms, containing at least one C═C double bond, e.g., ethenyl, propenyl, and the like.

“Alkoxy” refers to an alkyl group bonded to an oxygen atom. Alkoxy groups have the general formula: R—O.

“Antagonist” refers to a compound or a composition that attenuates the effect of an agonist. The antagonist can bind reversibly or irreversibly to a region of the receptor in common with an agonist. Antagonist can also bind at a different site on the receptor or an associated ion channel. Moreover, the term “antagonist” also includes functional antagonist or physiological antagonist. Functional antagonist refers to a compound and/or compositions that reverse the effects of an agonist rather than acting at the same receptor, i.e., functional antagonist causes a response in the tissue or animal which opposes the action of an agonist. Examples include agents which have opposing effects on an intracellular second messenger, or, on a physiologic state in an animal (for example, blood pressure). A functional antagonist can sometimes produce responses which closely mimic those of the pharmacological kind.

“Aryl” refers to a monovalent mono-, bi- or tricyclic aromatic hydrocarbon moiety of 6 to 15 ring atoms.

“Optionally-substituted aryl” refers to an aryl group as defined herein in which one or more aryl ring hydrogen is replaced with a non-hydrogen substituent such as halide, alkyl, cyano, hydroxy, alkoxy, etc. When two or more substituents are present in an aryl group, each substituent is independently selected.

“Biological activity” as used herein means having an effect on or eliciting a response from a living cell, tissue, organ or physiologic activity.

“Biomarker” as used herein means a measurable indicator of the severity or the presence of a particular disease state. More generally a biomarker is anything that can be used as an indicator of a particular disease state or some other physiological state of an organism.

“Chiral center” (i.e., stereochemical center, stereocenter, or stereogenic center) refers to an asymmetrically substituted atom, e.g., a carbon atom to which four different groups are attached. The ultimate criterion of a chiral center, however, is nonsuperimposability of its minor image.

“Cycloalkyl” refers to a non-aromatic, typically saturated, monovalent mono-, bi- or tri-cyclic hydrocarbon moiety of three to twenty ring carbons. The cycloalkyl can be optionally substituted with one or more, typically one, two, or three, substituents within the ring structure. When two or more substituents are present in a cycloalkyl group, each substituent is independently selected. Exemplary cycloalkyl groups include, but are not limited to, cyclopropyl, cyclobutyl, cyclopentyl, norbomyl, adamantyl, cyclohexyl, cyclooctyl, etc.

“Derivative” refers to a compound that is derived from some parent compound where one atom is replaced with another atom or group of atoms and usually maintains its general structure. For example, trichlormethane (chloroform) is a derivative of methane.

The terms “halo,” “halogen” and “halide” are used interchangeably herein and refer to fluoro, chloro, bromo, or iodo.

“Haloalkyl” refers to an alkyl group as defined herein in which one or more hydrogen atom is replaced by same or different halo atoms. The term “haloalkyl” also includes perhalogenated alkyl groups in which all alkyl hydrogen atoms are replaced by halogen atoms. Exemplary haloalkyl groups include, but are not limited to: —CH₂F, —CH₂Cl, —CF₃, —CH₂CF₃, —CH₂CCl₃, and the like.

“Hetero-substituted alkyl” refers to an alkyl group as defined herein that contains one or more heteroatoms such as N, O, or S. Such heteroatoms can be hydroxy, alkoxy, amino, mono- or di-alkyl amino, thiol, alkylthiol, etc.

“Hydroxyalkyl” refers to an alkyl group having one or more hydroxyl substituent(s).

“Enantiomeric excess” refers to the difference between the amounts of enantiomers. The percentage of enantiomeric excess (% ee) can be calculated by subtracting the percentage of one enantiomer from the percentage of the other enantiomer. For example, if the % of (R)-enantiomer is 99% and % of (S)-enantiomer is 1%, the % ee of (R)-isomer is 99%-1% or 98%.

“Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N, O-dimethylhydroxylamino, and the like.

“Ligand” as used herein means a biochemical substance in the form of a nucleic acid, protein or peptide that forms a complex with another biomolecule in a cell or tissue to serve a biological purpose.

“Moderate” as used herein means to decrease the quality, quantity, intensity or duration of a biological product or process.

“Pharmaceutically acceptable excipient” refers to an excipient that is useful in preparing a pharmaceutical composition that is generally safe, non-toxic, and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. A “pharmaceutically acceptable salt” of a compound also includes salts formed when an acidic proton present in the parent compound is either replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Pharmaceutically acceptable vehicle means, a carrier or inert medium used as a solvent (or diluent) in which the medicinally active agent is formulated and or administered.

The terms “pro-drug” and “prodrug” are used interchangeably herein and refer to any compound which releases an active parent drug according to Formula I or Formula 2 in vivo when such prodrug is administered to a mammalian subject. Prodrugs of a compound of Formula I or Formula 2 are prepared by modifying one or more functional group(s) present in the compound of Formula I or Formula 2 in such a way that the modification(s) may be cleaved in vivo to release the parent compound. Prodrugs include compounds of Formula I or Formula 2 wherein a hydroxy, amino, or sulfhydryl group in a compound of Formula I or Formula 2 is bonded to any group that may be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in compounds of Formula I or Formula 2, and the like. For example, the compound according to Formula 1 that is 4-({5-[ethyl(methyl)amino]-3-oxopentyl}oxy)benzoic acid can be reacted with CH₃CH₂OH under acidic conditions to produce: ethyl 4-({5-[ethyl(methyl)amino]-3-oxopentyl}oxy)benzoate, an ester prodrug that will be hydrolyzed to ethanol and the starting compound by esterase enzymes in tissues.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3rd edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

“Corresponding protecting group” means an appropriate protecting group corresponding to the heteroatom (i.e., N, O, P, or S) to which it is attached.

“Stereoisomer” means molecules that have the same molecular formula and sequence of bonded atoms (constitution), but differ in the three-dimensional orientations of their atoms in space. By definition, molecules that are stereoisomers of each other represent the same structural isomer. The chemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

“A therapeutically effective amount” means the amount of a compound that, when administered to an individual for treating a disease, is sufficient to effect such treatment for the disease. The “therapeutically effective amount” will vary depending on the compound, the disease and its severity or affected organ or tissue and the age, weight, etc., of the individual to be treated.

“Tautomer” or “tautomeric form” means structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons. The compounds of the present invention according to Formula 1 and Formula 2 can exist in different tautomer depend on the environment of the particular compound such as the acidity or alkalinity (i.e. pH) of the solution in which they are dissolved.

“Treating” or “treatment” of a disease means inhibiting the disease, i.e., arresting or reducing the pathophysiologic process or processes of the disease or its clinical symptoms; or relieving the disease, i.e., causing regression of the pathophysiologic process or processes of disease or reducing the clinical manifestations of the pathophysiologic process or processes of the specific disease.

### I. Methods of Synthesis

The compounds of Formula I or Formula 2 may be prepared by the methods described below, together with synthetic methods known in the art of organic chemistry, or modifications and transformations that are familiar to those of ordinary skill in the art. The starting materials used herein are commercially available or may be prepared by routine methods known in the art, such as those methods disclosed in standard reference books such as the Compendium of Organic Synthetic Methods, Vol. I-XII (eds. John Wiley & Sons, Inc., Hoboken, N.J., 2009). Preferred methods include, but are not limited to, those described below.

Preparation of the example compounds of Table 1 were prepared with standard procedures well known to those skilled in the art as follows:

All synthetic chemistry was performed in standard laboratory glassware unless indicated otherwise in the examples. Commercial reagents were used as received from the manufacturer. Analytical LC/MS was performed on an Agilent 1200 system with a variable wavelength detector and Agilent 6140 single quadrupole mass spectrometer, alternating positive and negative ion scans. Retention times were determined from the extracted 220 nm UV chromatogram. Preparative HPLC was performed on a Wufeng LC120 system (Shanghai Wufeng Scientific Instruments Co., Ltd. Shanghai., China). 1H NMR was performed on a Bruker AVANCE™ 300 at 300 MHz or a Bruker AVANCE™ DRX 500 at 500 MHz. For complicated splitting patterns, the apparent splitting is tabulated. Analytical thin layer chromatography was performed on silica (Macherey-Nagel ALUGRAM® Xtra SIL G, 0.2 mm, UV254 indicator) and was visualized under UV light. Silica gel chromatography was performed manually, or with an Isco COMBIFLASH® for gradient elutions.

Analytical LC/MS method: HPLC column: Kinetex, 2.6 μm, C18, 50×2.1 mm, maintained at 40° C. HPLC Gradient: 1.0 mL/min, 95:5:0.1 water:acetonitrile:formic acid to 5:95:0.1 water:acetonitrile:formic acid in 2.0 min, maintaining for 0.5 min. Preparative HPLC method: HPLC Gradient: 100 mL/min, 95:5:0.1 acetonitrile:water:trifluoroacetic acid to 70:30:0.1 acetonitrile:water:trifluoroacetic acid in 9.5 min, maintaining for 0.5 min.

### Compound 1: 4-[3-[2-(Dimethylamino)ethylamino]-3-oxopropoxy]benzoic Acid

A solution of 3-(4-(methoxycarbonyl)phenoxy)propanoic acid (150 mg, 0.67 mmol) in thionyl chloride (750 μL, 10.32 mmol) was stirred at 50° C. for 1 h. The reaction mixture was concentrated under a nitrogen stream to remove thionyl chloride. The residue was dissolved in chloroform (1 mL) and evaporated under a nitrogen stream. The residue was dissolved in chloroform (1 mL) and added dropwise to a stirred solution of N,N-dimethylethylenediamine (66 μL, 0.60 mmol) in chloroform (1 mL) at room temperature. The reaction mixture was stirred at room temperature for 18 h. The reaction mixture was washed with saturated sodium bicarbonate (1 mL). The aqueous layer was extracted with chloroform (1 mL). The combined organic layers were dried over sodium sulfate and evaporated to give Methyl 4-[3-[2-(dimethylamino)ethylamino]-3-oxopropoxy]benzoate (107 mg, 55%) as a white crystalline solid. Next, A mixture of methyl 4-(3-((2-(dimethylamino)ethyl)amino)-3-oxopropoxy)benzoate (173 mg, 0.59 mmol) and lithium hydroxide (1 N aqueous solution, 865 μL, 865 mmol) in 1,4-dioxane (865 μL) was stirred at room temperature for 1 h. The reaction mixture was neutralized to pH 7 by addition of acetic acid. The mixture was concentrated to 1 mL, washed with chloroform (1 mL) and evaporated. The crude product was purified by preparative HPLC. The pure fractions were combined and lyophilized. The residue was dissolved in water (20 mL) and to this solution was added 1 N hydrochloric acid (500 μL). The solution was evaporated. The residue was dissolved in water (3×10 mL) and evaporated. The residue was dissolved in water (5 mL) and lyophilized to yield Compound 1: 4-[3-[2-(Dimethylamino)ethylamino]-3-oxopropoxy]benzoic acid (45 mg, 24%) as a white crystalline solid.

LCMS: 99%, tR=0.243 min, m/z=281 [M+H]+, Method: BB_LCMS_05_KINETEX.

1H NMR (300 MHz, DMSO-d6) δ 12.61 (br s, 1H), 10.24 (br s, 1H), 8.48-8.32 (m, 1H), 7.88 (d, J=8.8 Hz, 2H), 7.00 (d, J=8.9 Hz, 2H), 4.28 (t, J=6.1 Hz, 2H), 3.52-3.40 (m, 2H), 3.19-3.07 (m, 2H), 2.77 (s, 6H), 2.63 (t, J=6.1 Hz, 2H).

### Compound 7: N-[2-(Diethylamino)ethyl]-3-(4-methoxyphenoxy)propanamide

A mixture of 3-(4-methoxyphenoxy)propanoic acid (100 mg, 0.51 mmol) in thionyl chloride (500 μL, 6.88 mmol) was stirred at room temperature for 1.5 h. The reaction mixture was concentrated under a nitrogen stream to remove thionyl chloride. The residue was dissolved in chloroform (1 mL) and evaporated under a nitrogen stream. The residue was dissolved in chloroform (1 mL) and added dropwise to a stirred solution of N,N-diethylethylenediamine (66 μL, 0.46 mmol) in chloroform (1 mL) at room temperature. The reaction mixture was stirred at room temperature for 30 min. The mixture was washed with saturated sodium bicarbonate (1 mL). The aqueous layer was extracted with chloroform (1 mL) and the combined organic layers were dried over sodium sulfate and evaporated. The crude product (108 mg) was dissolved in 1 N hydrochloric acid (5 mL) and the aqueous layer was washed with diethyl ether (5 mL). The aqueous layer was made basic to pH 8 by addition of sodium bicarbonate and extracted with diethyl ether (5 mL). The organic layer was dried over sodium sulfate and evaporated to give Compound 7: N-[2-(diethylamino)ethyl]-3-(4-methoxyphenoxy)propanamide (89 mg, 59%) as a pale yellow oil.

LCMS: 99%, tR=0.728 min, m/z=295 [M+H]+, Method: BB_LCMS_05_KINETEX. 1H NMR (300 MHz, DMSO-d6) δ 7.84 (t, J=5.7 Hz, 1H), 6.87-6.80 (m, 4H), 4.09 (t, J=6.2 Hz, 2H), 3.69 (s, 3H), 3.16-3.06 (m, 2H), 2.53-2.38 (m, 8H), 0.94 (t, J=7.1 Hz, 6H).

### Compound 12: N-[2-(Diethylamino)ethyl]-N-ethyl-3-(4-methoxyphenoxy)propanamide

N-[2-(Diethylamino)ethyl]-N-ethyl-3-(4-methoxyphenoxy)propanamide was prepared by the method used for compound 7 above, starting with 3-(4-methoxyphenoxy)propanoic acid and N,N,N′-triethylenediamine.

LCMS: 99%, tR=0.976 min, m/z=323 [M+H]+, Method: BB_LCMS_05_KINETEX.

1H NMR (300 MHz, Chloroform-d) δ 6.90-6.80 (m, 4H), 4.33-4.25 (m, 2H), 3.78 (s, 3H), 3.51-3.33 (m, 4H), 2.81 (t, J=6.7 Hz, 2H), 2.68-2.50 (m, 6H), 1.22 (t, J=7.1 Hz, 3H), 1.06 (t, J=7.3 Hz, 6H).

### Compound 16: 3-(4-Methoxyphenoxy)-N-[2-(morpholin-4-yl)ethyl]propanamide

3-(4-Methoxyphenoxy)-N-[2-(morpholin-4-yl)ethyl]propanamide was prepared by the method used for compound 7 above, starting with 3-(4-methoxyphenoxy)propanoic acid and 2-morpholinoethanamine.

LCMS: 98%, tR=0.444 min, m/z=309 [M+H]+, Method: BB_LCMS_05_KINETEX.

1H NMR (300 MHz, DMSO-d6) δ 7.88 (t, J=5.7 Hz, 1H), 6.88-6.80 (m, 4H), 4.09 (t, J=6.2 Hz, 2H), 3.69 (s, 3H), 3.60-3.49 (m, 4H), 3.24-3.13 (m, 2H), 2.53-2.44 (m, 2H), 2.42-2.29 (m, 6H).

### Compound 17: N-[2-(Diethylamino)ethyl]-3-(3-fluoro-4-methoxyphenoxy)propanamide

A mixture of 3-fluoro-4-methoxyphenol (250 mg, 1.76 mmol), acrylonitrile (1.2 mL, 17.6 mmol), potassium carbonate (12 mg, 0.09 mmol) and tert-butanol (17 μL, 0.18 mmol) was stirred at 75° C. for 16 h in a sealed tube. To the reaction mixture was added potassium carbonate (12 mg, 0.09 mmol) and the stirring was continued at 75° C. for 8 h. The reaction mixture was evaporated. The residue was taken up in toluene (2 mL) and washed with 10% aqueous sodium hydroxide (1 mL). The aqueous layer was extracted with toluene (2 mL). The combined organic layers were washed with 10% aqueous potassium bisulfate (1 mL), dried over sodium sulfate and evaporated to give 3-(3-fluoro-4-methoxyphenoxy)propanenitrile (231 mg, 67%) as an off-white solid. Next, a mixture of 3-(3-fluoro-4-methoxyphenoxy)propanenitrile (222 mg, 1.14 mmol) in concentrated hydrochloric acid (1 mL) and water (500 μL) was stirred at 100° C. for 3 h. The reaction mixture was poured onto ice (5 g) and the mixture was stirred for 15 min. The precipitate was collected, washed with water (2 mL) and dried in air. The crude product (196 mg) was dissolved in 10% aqueous sodium carbonate (10 mL) and the aqueous layer was washed with dichloromethane (10 mL). The aqueous layer was acidified to pH 1 by addition of 1 N hydrochloric acid. The precipitate was collected, washed with water (2×1 mL) and dried in air to give 3-(3-Fluoro-4-methoxyphenoxy)propanoic acid (103 mg, 42%) as an off-white solid. Finally, a mixture of 3-(3-Fluoro-4-methoxyphenoxy)propanoic acid (100 mg, 0.51 mmol) in thionyl chloride (500 μL, 6.88 mmol) was stirred at room temperature for 1.5 h. The reaction mixture was concentrated under a nitrogen stream to remove thionyl chloride. The residue was dissolved in chloroform (1 mL) and evaporated under a nitrogen stream. The residue was dissolved in chloroform (1 mL) and added dropwise to a stirred solution of N,N-diethylethylenediamine (66 μL, 0.46 mmol) in chloroform (1 mL) at room temperature. The reaction mixture was stirred at room temperature for 30 min. The mixture was washed with saturated sodium bicarbonate (1 mL). The aqueous layer was extracted with chloroform (1 mL) and the combined organic layers were dried over sodium sulfate and evaporated. The crude product (108 mg) was dissolved in 1 N hydrochloric acid (5 mL) and the aqueous layer was washed with diethyl ether (5 mL). The aqueous layer was made basic to pH 8 by addition of sodium bicarbonate and extracted with diethyl ether (5 mL). The organic layer was dried over sodium sulfate and evaporated to give Compound 17: N-[2-(diethylamino)ethyl]-3-(3-fluoro-4-methoxyphenoxy)propanamide (54 mg, 59%) as a pale yellow oil.

1H NMR (300 MHz, Chloroform-d) δ 6.89 (t, J=9.2 Hz, 1H), 6.73 (dd, J=12.6, 2.9 Hz, 1H), 6.67-6.60 (m, 1H), 6.67-6.56 (m, 1H), 4.21 (t, J=6.0 Hz, 2H), 3.86 (s, 3H), 3.40-3.30 (m, 2H), 2.66 (t, J=6.0 Hz, 2H), 2.61-2.51 (m, 6H), 1.03 (t, J=7.1 Hz, 6H).

### II. Methods of Inhibition

The compounds and compositions described herein moderate biological activity of one or more protein components of the HMGB1 pathway and/or MyD88 and RAGE mediated signaling activity and transduction. In addition to inhibiting the transduction of biochemical signals in cells or tissues caused by the interaction of High Mobility Group Protein B1 and its ligands, the compounds and compositions described also moderate later portions of the inflammatory process by either decreasing the genetic expression of relevant ligands, protein kinases, and transcription factors or by moderating the physical and/or the chemical interactions of the relevant ligands involved to reduce the pro-inflammatory cascade induced by HMGB1 through its interaction with TLR2/TLR4 and RAGE receptors. Non-limiting examples are down-regulation of NF-kB (nuclear factor kappa-light chain enhancer of activated B cells) gene expression and/or reducing the physical association of two ligands with each other. The compounds and compositions described are also useful for moderating several signaling pathways that are activated in the course of inflammation induced by HMGB1.

In addition, the compounds and compositions described are useful in moderating monocyte migration and T-cell and Dendritic cell interaction through reduction of chemokines such as CCL2 as well as moderating signaling via other pro-inflammatory mechanisms such as VCAM-1 induced adhesion of inflammatory cells to the vascular endothelium at the site of injury.

### III. Methods of Treatment

Because HMGB1 is involved in the inflammation response and because it acts as a cytokine, management of HMGB1 expression and activity has implications for disease treatment. Studies have indicated that antibodies which neutralize HMGB1 confer protection against damage and tissue injury during arthritis, colitis, ischemia, sepsis, endotoxernia, and systemic lupus erythematosus. In addition, and as non-limiting examples, inflammation is associated with the following diseases/disorders: autoimmune diseases; inflammatory diseases; autoinflammatory conditions; pain conditions; respiratory; airway and pulmonary conditions; gastrointestinal disorders; allergic diseases; infection-based diseases; trauma and tissue injury-based conditions; fibrotic diseases; ophthalmic/ocular diseases; joint, muscle and bone disorders; skin/dermatological diseases; renal diseases; genetic diseases; hematopoietic diseases; liver diseases; oral diseases; metabolic diseases, including diabetes (e.g. Type II) and complications thereof; proliferative diseases; cardiovascular conditions; vascular conditions; neuro-inflammatory conditions; neurodegenerative conditions; cancer; sepsis; pulmonary inflammation and injury; or pulmonary hypertension.

Increased tissue or blood levels of HMGB1 is directly contributory to the pathogenesis of acute and chronic inflammatory diseases, through its interaction with TLR2/TLR4 and RAGE receptor and subsequent downstream signaling. Such diseases include, but are not limited to: acute pancreatitis, asthma, atopic dermatitis, chronic hepatitis, coronary artery disease, granulomatous nephritis, inflammatory bowel disease, Parkinson's disease, psoriasis, rheumatoid arthritis, rhino-sinusitis, systemic lupus erythematosus, and tissue hypoxia-reperfusion injury.

HMGB1 increases the levels of pro-inflammatory cytokines and chemokines through its interaction with TLR2/TLR4 and RAGE receptors and subsequent downstream signaling pathway effects such as, but not limited to: IL-1 beta, TNF-alpha, IL-6, IL-17 and IL-23. These increased pro-inflammatory cytokines and chemokines induce the pathogenic processes responsible for acute and chronic inflammatory diseases such as; but not limited to; acute pancreatitis, asthma, atopic dermatitis, chronic hepatitis, coronary artery disease, granulomatous nephritis, inflammatory bowel disease, Parkinson's disease, psoriasis, rheumatoid arthritis, rhino-sinusitis, systemic lupus erythematosus and tissue hypoxia-reperfusion injury.

HMGB1, through its interaction with TLR2/TLR4 and RAGE receptors, also induces increased gene expression of specific protein kinases, transcription factors, G-Protein Coupled Receptors (GPCRs), and other biological ligands important to downstream pro-inflammatory signaling. Examples, include but are not limited to: p38 MAPK (Entrez Gene: 1432), NF-κB (Entrez Gene: 4790), CCL2 (Entrez Gene: 6347), TLR2 (Entrez Gene: 7097), VCAM (Entrez Gene: 7412), Myeloid differentiation primary response protein 88 (Entrez Gene: 4615). Up regulation and increased expression of these genes contribute to the disease process of several inflammatory diseases, such as but not limited to; acute pancreatitis, asthma, atopic dermatitis, chronic hepatitis, chronic inflammatory demyelinating polyradicular neuritis, coronary artery disease, dermatomyositis, granulomatous nephritis, inflammatory bowel disease, Parkinson's disease, periodontitis, psoriasis, rheumatoid arthritis, rhino-sinusitis, systemic lupus erythematosus, tissue hypoxia-reperfusion injury, and ulcerative colitis.

A method of treatment comprises the administration of one or more therapeutic agents according to Formula 1 and/or Formula 2 for treatment of one or more diseases. Combinations of therapeutic agents can be used to treat one disease or multiple diseases or to moderate the side-effects of one or more agents in the combination. The compounds described herein can be used in combination with other treatment agents. Non-limiting examples of other treatment agents are: non-steroidal anti-inflammatory drugs; immunomodulatory and/or anti-inflammatory agents; antimalarials; antibiotics; Anti-TNFα agents; Anti-CD20 agents; Antidiarrheals; Bile acid binding agents; laxatives; T lymphocyte activation; Anti-IL1 treatments; Glucocorticoid receptor modulators; Aminosalicyic acid derivatives including but not limited to: sulfasalazine and mesalazine; Anti-α4 integrin agents; α1- or α2-adrenergic agonist agents; β-adrenergic agonists; Anticholinergic agents; inhaled long acting beta-agonists; long acting muscarinic antagonists; long acting corticosteroids; leukotriene pathway modulators; and H1 receptor antagonists. Where the compounds disclosed herein are administered in conjunction with other agents, dosages of the co-administered agents will of course vary depending on the type of co-drug employed, e.g. whether it is a steroid or a calcineurin inhibitor, on the specific drug employed, on the condition being treated, and so forth.

In an embodiment of the present invention the compounds according to Formula 1 and Formula 2 may be used either simultaneously or sequentially in combination with, a second compound, including those listed below.

Non-steroidal anti-inflammatory drugs, such as but not limited to: aspirin, choline salicylate, celecoxib, acetaminophen, diclofenac, flurbiprofen, ibuprofen, indomethacin, ketoprofen, meclofenamate, mefenamic acid, nabumetone, naproxen, piroxicam, rofecoxib, salicylates, sulindac, tolmetin, and valdecoxib.

Immunomodulatory agents, such as but not limited to: methotrexate, azathioprine, mitoxantrone, cladribin, cyclophosphamide, tacrorimus, cyclosporine, and hydroxychloroquine.

Antimalarials, such as but not limited to: chloroquine, quinine, amodiaquine, pyrimethamine, proguanil, mefloquine, atovaquone, primaquine, artemisinin, and halofantrine.

Antibiotics, such as but not limited to: minocycline, doxycycline, sulfonamides, and clindamycin.

Anti-TNF alpha agents, such as but not limited to: infliximab, adalimumab, certolizumab pegol, golimumab, thalidomide, lenalidomide, pomalidomide, and etanercept.

Anti-CD20 agents, such as but not limited to: rituximab, obinutuzumab, Ibritumomab tiuxetan, and tositumomab.

Antidiarrheals, such as but not limited to; lidamidine, diphenoxylate, loperamide, and quercetin.

T lymphocyte activation inhibitors, such as but not limited to: voclosporin, peroxynitrite, and dasatinib.

Anti-IL-1 agents, such as but not limited to: anakinra and IL-1Ra.

Glucocorticoids, such as but not limited to: methyl prednisolone, prednisolone, dexamethasone, betamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl) ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester, and 6α,9α-difluoro-11β-hydroxy-16α-methyl-17α-(1-ethycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone furoate, triamcinolone acetonide, rofleponide, ciclesonide (16α,17-[[(cis)-cyclohexylmethylene]bis(oxy)]-11β,21-dihydroxy-pregna-1,4-diene-3,20-dione), butixocort propionate, RPR-106541, and ST-126.

Sex steroids and receptor modulators, such as but not limited to: progesterone, progestins, androgen, estrogen, mifepristone and misoprostil.

Aminosalicylic acid derivatives such as but not limited to: sulfasalazine and mesalazine.

Anticholinergic agents, such as but not limited to: compounds that act as antagonists at the muscarinic receptors, in particular those compounds which are antagonists of the M₁ or M3 receptors, dual antagonists of the M1/M3 or M2/M3, receptors or pan-antagonists of the M1/M2/M3 receptors. For example; ipratropium, oxitropium, tiotropium, revatropate, pirenzepine, darifenacin, oxybutynin, terodiline, tolterodine, otilonium, trospium chloride, and solifenacin.

Beta adrenergic agonists, such as but not limited to: salmeterol, salbutamol, formoterol, salmefamol, fenoterol, carmoterol, etanterol, naminterol, clenbuterol, pirbuterol, flerbuterol, reproterol, bambuterol, indacaterol, terbutaline, and salts thereof, for example the xinafoate (1-hydroxy-2-naphthalenecarboxylate) salt of salmeterol.

Corticosteroids, such as but not limited to: cortisone and hydrocortisone.

Phosphodiesterase inhibitors, specifically phosphodiesterase 4 (PDE4) inhibitors such as but not limited to: mesembrenone, rolipram, Ibudilast, piclamilast, luteolin, drotaverine, roflumilast, cilomilast, and apremilast.

Leukotriene pathway modulators, such as but not limited to: 3-[3-butylsulfanyl-1-[(4-chlorophenyl)methyl]-5-propan-2-yl-indol-2-yl]-2,2-dimethyl-propanoic acid, baicalein, caffeic acid, curcumin, hyperforin, and zileuton.

Histamine receptor antagonists, such as but not limited to: melexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, cetirizine, levocetirizine, efletirizine, fexofenadine, exofenadine, cimetidine, ranitidine, famotidine, nizatidine, (1-[(5-Chloro-1H-benzimidazol-2-yl)carbonyl]-4-methylpiperazine), 5-chloro-2-[(4-methylpiperazin-1-yl)carbonyl]-1H-indole, and thioperamide.

Administration of the therapeutic agent may be by any suitable means. In some embodiments, the one or more therapeutic agents are administered by oral administration. In some embodiments, the one or more therapeutic agents are administered by transdermal administration. In some embodiments, the one or more therapeutic agents are administered by injection or intravenous infusion. In one embodiment, the one or more therapeutic agents are administered topically to a mucosal, dermal or ocular tissue.

If combinations of agents are administered as separate compositions, they may be administered by the same route or by different routes. If combinations of agents are administered in a single composition, they may be administered by any suitable route. In some embodiments, combinations of agents are administered as a single composition by oral administration. In some embodiments, combinations of agents are administered as a single composition by transdermal administration. In some embodiments, the combinations of agent are administered as a single composition by injection. In some embodiments, the combinations of agent are administered as a single composition topically.

In one embodiment of the present invention the compounds of Formula 1 and Formula 2 may contain asymmetric or chiral centers, and, therefore, exist in different stereoisomeric forms. For example, N-[2-(diethylamino)ethyl]-N-ethyl-3-(4-methoxyphenoxy)propanamide a compound according Formula 1 that possesses a chiral center at the second nitrogen atom and thus has two stereoisomer forms. It is intended that all stereoisomeric forms of the compounds of Formula I and Formula 2 form part of the present invention, including but not limited to: diastereomers, enantiomers, and atropisomers as well as mixtures thereof such as racemic mixtures. In addition, the present invention embraces all geometric and positional isomers. For example, if a compound of Formula I or Formula 2 incorporates a double bond or a fused ring, both the cis- and trans-forms, as well as mixtures, are embraced within the scope of the invention. Both the single positional isomers and mixture of positional isomers are also within the scope of the present invention.

In one embodiment of the present invention, compounds of Formula 1 and Formula 2 may exist in different tautomeric forms, and all such forms are embraced within the scope of the invention, as defined by the claims.

In one embodiment of the present invention the therapeutically effective dose is from about 0.01 mg to about 2,000 mg per day of a compound provided herein. The pharmaceutical compositions therefore should provide a dosage of from about 0.01 mg to about 2000 mg of the compound. In certain embodiments, pharmaceutical dosage unit forms are prepared to provide from about 1 mg to about 2000 mg, from about 10 mg to about 1000 mg, from about 20 mg to about 500 mg or from about 25 mg to about 250 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

### IV. Examples

To determine the ability of compounds according to Formula 1 and Formula 2 to moderate HMGB1 signal transduction through TLR2/TLR4 (MyD88 dependent pathway) and through RAGE (MyD88 independent pathway) using a functional assay, cultivated human monocytes or PBMCs were treated with purified human recombinant HMGB1 protein and the expression of various inflammatory cytokines and other genes known to be upregulated by HMGB1, were determined by well-known methods as described below.

Freshly isolated Human PBMCs or CD14+ monocytes from healthy volunteers were isolated and cultured at 1×10⁶ cell/ml in RPMI-1640 medium (GIBCO® Inc. Carlsbad, Calif., USA) supplemented with 20% fetal bovine serum and 1% streptomycin/penicillin. The cell suspensions were plated in 6-well culture plates and stimulated with 0.5 μg/ml of HMGB1 (INVITROGEN™ Inc., Carlsbad Calif.) for 18 hrs. Cell suspensions without HMGB1 stimulation were used as a baseline control for cytokine production. Cell cultures were treated with various exemplar compounds according to Formula 1 and Formula 2 at several concentrations. As a known positive control for TLR2/TLR4 and RAGE pathway inhibitory moderation, a blocking antibody to human HMGB1 (R&D SYSTEMS®, Minneapolis, Minn. USA) was used (10 ug/ml) that prevents binding of HMGB1 to TLR2/TLR4 and RAGE receptors and therefore blocks signal transduction through the TLR2/TLR4 (MyD88 dependent) and RAGE (Myd88 independent) signaling pathways. As a known positive control for RAGE pathway specific (MyD88 independent) signaling, a blocking antibody to TLR2/TLR4 (R&D SYSTEMS®, Minneapolis, Minn. USA) was used (10 ug/ml) prior to addition of HMGB1, thus enabling detection of only RAGE pathway (MyD88 independent) induced changes in gene expression and cytokine production. In this fashion, differential activity of HMGB1, MyD88 dependent, and MyD88 independent signaling was detected.

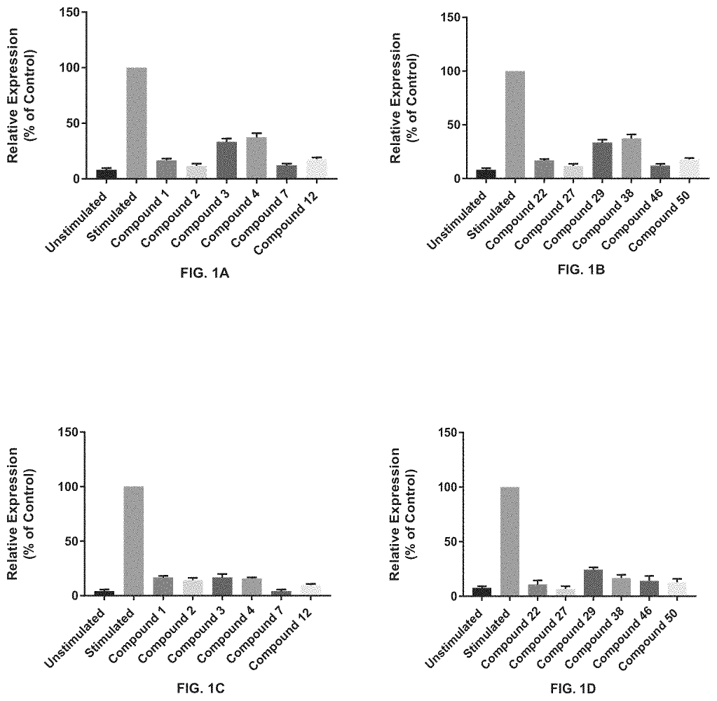

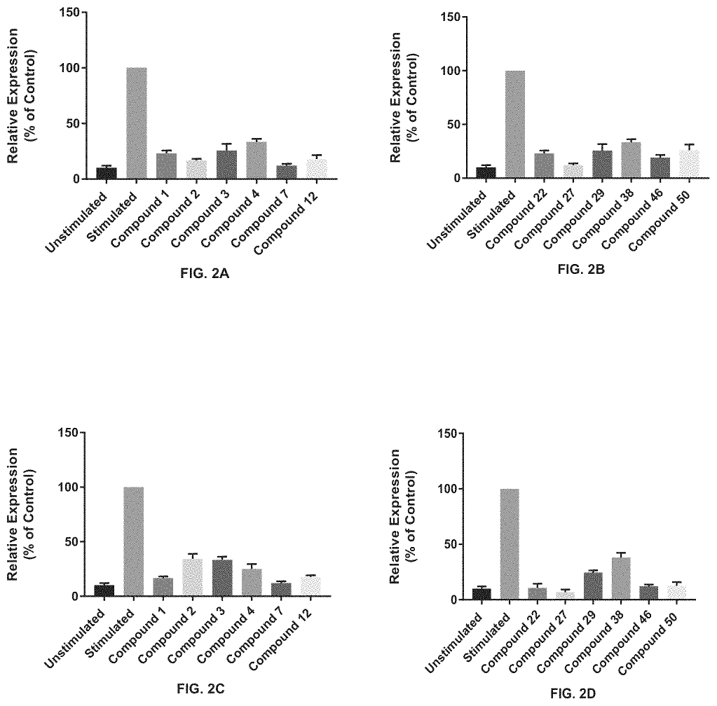

The expression of specific genes known to be up-regulated by HMGB1, either through TLr2/TLR4 receptors (MyD88 dependent signaling) or the RAGE receptor (MyD88 independent signaling), were measured by quantitative real-time polymerase chain reaction (qRT-PCR), using RT²™ qPCR Primer Assays (QIAGEN®, Valencia, Calif., USA). Cell cultures were incubated for 6 hrs with compounds of the invention prior to extraction of RNA using TRIZOL® reagent (THERMOFISHER SCIENTIFIC™, San Diego, Calif., USA). All sample assays were standardized to the expression level two known human “housekeeping genes” using RT² qPCR Primer Assays (QIAGEN®, Valencia, Calif., USA) according to manufacturer's specifications. These “housekeeping genes,” i.e. ribosomal protein large P1 (RPLP1) and glyceraldehyde-3-phosphate dehydrogenase (GAPDH), are constitutively expressed and not upregulated by HMGB1. All genes in the assay were amplified in triplicate. Examples of the results of these experiments are shown in FIG. 1A-D and FIG. 2A-D. These experiments demonstrate that the increased expression of specific downstream genes associated with TLR2/TLR4 (MyD88 dependent) signaling and RAGE (MyD88 independent) signaling induced by HMGB1 are inhibited by the compounds of the instant invention.

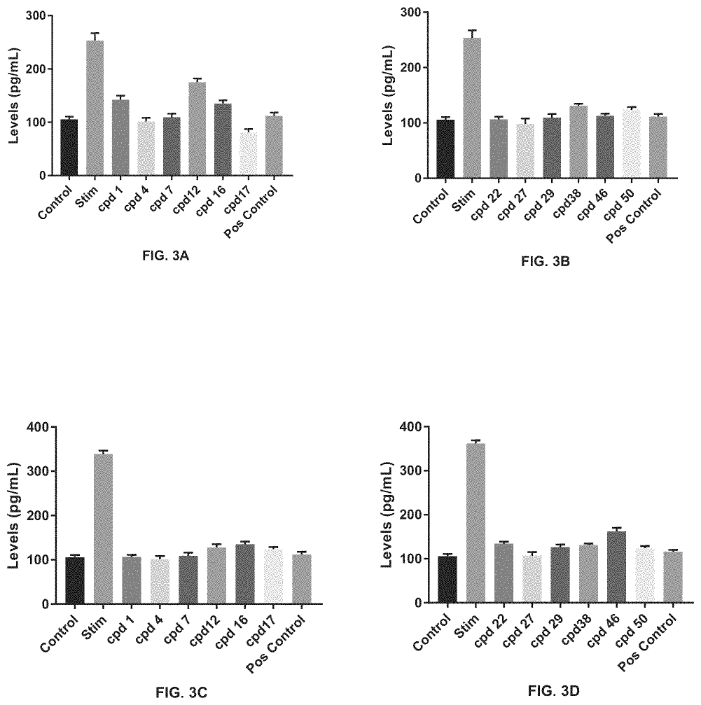

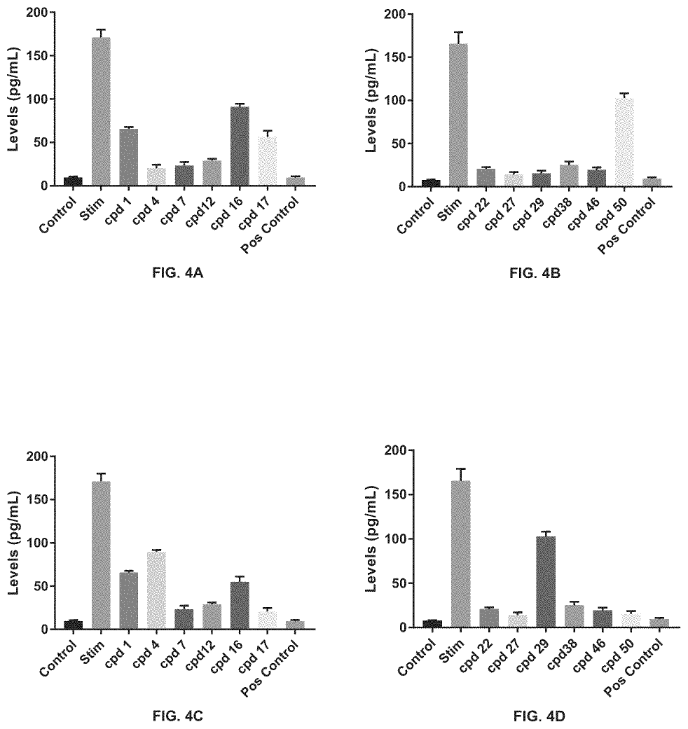

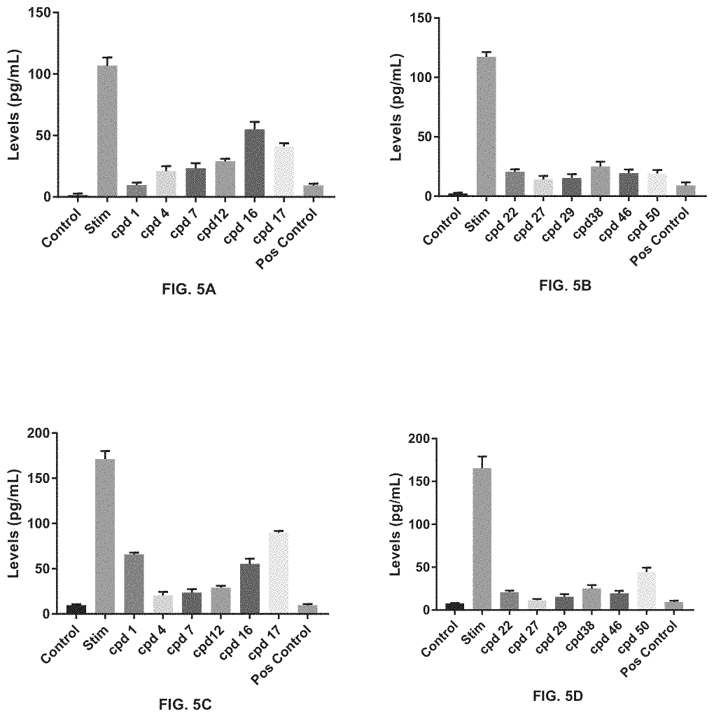

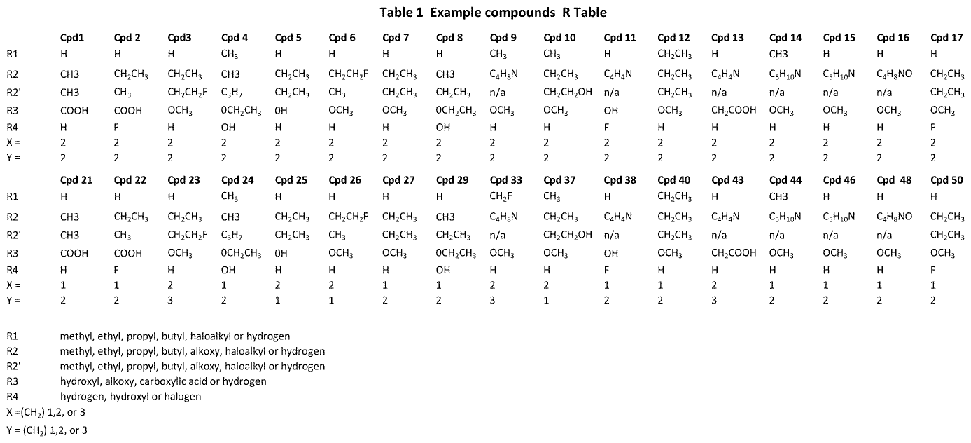

The concentration of secreted cytokines known to be increased by HMGB1 exposure in monocytes and PBMCs through the TLR2/TLR4 pathway (i.e. IL-6, TNF-alpha, and IL17) were measured. Using commercially obtained enzyme-linked immunosorbent assay (ELISA) kits (R&D SYSTEMS®, Minneapolis, Minn. USA) according to the manufacturer's instructions, the secreted cytokines in the supernatant from cell cultures stimulated with HMGB1 alone or treated with various compounds was measured along with the positive controls. The concentration of cytokines known to be increased by HMGB1 in monocytes and PBMCs through RAGE pathway (i.e. IL-23, MCP-1/CCL2) were similarly measured in the supernatant from cell cultures stimulated with HMGB1 alone or treated with various compounds along with the positive control. Cell suspensions without HMGB1 stimulation were used as baseline control for cytokine production. Experiments were performed in triplicate. Examples of the results of these experiments are shown in FIGS. 2A and 2B, FIGS. 3A and 3Bb, and FIGS. 4A and 4B. Due to the high levels of some cytokines, cell culture supernatants were diluted to enable the values to fall on the exponential phase of the standard curve. The calculated values were therefore multiplied by this dilution factor.

The results of the experiments herein demonstrate that compounds according to Formula 1 and Formula 2 that are the subject matter of the instant invention inhibit the pro-inflammatory activity of HMGB1 by decreasing the production of HMGB1 induced pro-inflammatory cytokines as well as decreasing the expression of other genes associated with the HMGB1 induced inflammatory cascades in human immune cells. Therefore, the compounds according to Formula I are useful in treatment of various disorders associated with HMGB1 induced inflammation.

### REFERENCES

[1] Bianchi M E, Beltrame M, Paonessa G. Specific recognition of cruciform DNA by nuclear protein HMG1. Science 1989; 243:1056-9.
[2] Bustin M. Revised nomenclature for high mobility group (HMG) chromosomal proteins. Trends Biochem Sci 2001; 26:152-3.
[3] Goodwin G H, Sanders C, Johns E W. A new group of chromatin associated proteins with a high content of acidic and basic amino acids. Eur J Biochem 1973; 38:14-9.
[4] Andersson U, Wang H, Palmblad K, Aveberger A C, Bloom O, Erlandsson-Harris H et al. High mobility group 1 protein (HMG-1) stimulates proinflammatory cytokine synthesis in human monocytes. J Exp Med 2000; 192:565-70.
[5] Li K, Kokkola R, Tabibzadeh S, et al. Structure basis for the proinflammatory cytokine activity of high mobility group box 1. Mol. Med. 2003, 9:37-45.
[6] Yang H, Tracey K J. Targeting HMGB1 in inflammation. Biochimica et Biophysica Acta (BBA) 2010; 1799:149-156.
[7] Takeda K, and Akira S. TLR signaling pathways. Seminars in Immunology (2004) 16:3-9.
[8] Riehl A, Nemeth J, et al. The receptor RAGE: Bridging inflammation and cancer. Cell Communication and Signaling 2009, 7:12.

1.-25. (canceled)

26. A compound according to Formula 1 below: 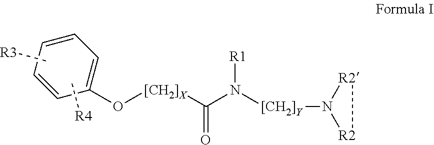
wherein
X=1-3;
Y=1-3;
R1=is independently selected from methyl, ethyl, propyl, butyl, haloalkyl, or hydrogen;
R2=is independently selected from methyl, ethyl, propyl, butyl, alkoxy, haloalkyl, or hydrogen;
R2′=is independently selected from methyl, ethyl, propyl, butyl, alkoxy, haloalkyl, or hydrogen;
R2 & R2′ may be fused into a heterocyclic ring with 5 or 6 members;
R3=is independently selected from methyl, ethyl, hydroxyl, alkoxy, carboxylic acid, or haloalkyl; and
R4=is independently selected from hydrogen, hydroxyl or halogen,
or a pharmaceutically acceptable salt, addition compound or pro-drug thereof.

27. A pharmaceutical composition comprising a compound of claim 26 or a pharmaceutically acceptable salt thereof or a tautomer of said compound or said salt and a pharmaceutically acceptable vehicle, diluent or carrier.

28. A pharmaceutical combination comprising a therapeutically effective amount of a composition comprising:
(a) a first compound according to claim 26 or a pharmaceutically acceptable salt thereof; and
(b) a second compound selected from the group consisting of non-steroidal anti-inflammatory drugs, immunomodulatory and/or anti-inflammatory agents, antimalarials, antibiotics, Anti-TNFα agents, Anti-CD20 agents, Antidiarrheals, Bile acid binding agents, laxatives, T lymphocyte activation inhibitors, Anti-IL1 treatments, Glucocorticoids, Steroid receptor modulators, Aminosalicylic acid derivatives Anti-α4 integrin agents, adrenergic agonist agents, anticholinergic agents, inhaled long acting beta-agonists, long acting muscarinic antagonists, long acting corticosteroids, leukotriene pathway modulators, and H1 receptor antagonists.

29. A compound selected from the group consisting of: 4-[3-[2-(Dimethylamino)ethylamino]-3-oxopropoxy]benzoic acid; 4-[2-({2-[ethyl(methyl)amino]ethyl}carbamoyl)ethoxy]-2-fluorobenzoic acid; 4-[2-({2-[(2-fluoroethyl)(methyl)amino]ethyl}carbamoyl)ethoxy]benzoic acid; 3-(4-ethoxy-3-hydroxyphenoxy)-N-methyl-N-{2-[methyl(propyl)amino]ethyl}propanamide; N-[2-(diethylamino)ethyl]-3-(3-hydroxyphenoxy)propanamide; N-{2-[(2-fluoroethyl)(methyl)amino]ethyl}-3-(3-methoxyphenoxy)propanamide; N-[2-(Diethylamino)ethyl]-3-(4-methoxyphenoxy)propanamide; N-{2-[ethyl(methyl)amino]ethyl}-3-(3-methoxyphenoxy)propanamide; 3-(3-methoxyphenoxy)-N-methyl-N-[2-(pyrrolidin-1-yl)ethyl]propanamide; N-{2-[ethyl(2-hydroxyethyl)amino]ethyl}-3-(3-methoxyphenoxy)-N-methylpropanamide; 3-(3-fluoro-5-hydroxyphenoxy)-N-[2-(1H-pyrrol-1-yl)ethyl]propanamide; N-[2-(diethylamino)ethyl]-N-ethyl-3-(4-methoxyphenoxy)propanamide; 2-[3-(2-{[2-(1H-pyrrol-1-yl)ethyl]carbamoyl}ethoxy)phenyl]acetic acid; 3-(3-methoxyphenoxy)-N-methyl-N-[2-(piperidin-1-yl)ethyl]propanamide; 3-(3-methoxyphenoxy)-N-[2-(piperidin-1-yl)ethyl]propanamide; 3-(4-methoxyphenoxy)-N-[2-(morpholin-4-yl)ethyl]propanamide; N-[2-(diethylamino)ethyl]-3-(3-fluoro-4-methoxyphenoxy)propanamide; 4-({[2-(dimethylamino)ethyl]carbamoyl}methoxy)benzoic acid; 3-[({2-[ethyl(methyl)amino]ethyl}carbamoyl)methoxy]-5-fluorobenzoic acid; N-{3-[ethyl(2-fluoroethyl)amino]propyl}-2-(4-methoxyphenoxy)acetamide; 2-(3-hydroxy-4-methoxyphenoxy)-N-methyl-N-{2-[methyl(propyl)amino]ethyl}acetamide; N-[(diethylamino)methyl]-3-(4-hydroxyphenoxy)propanamide; N-{[(2-fluoroethyl)(methyl)amino]methyl}-2-(4-methoxyphenoxy)acetamide; N-(2-(diethylamino)ethyl)-2-(4-methoxyphenoxy)acetamide; N-{2-[ethyl(methyl)amino]ethyl}-2-[4-(methoxymethyl)phenoxy] acetamide; 3-(4-methoxyphenoxy)-N-methyl-N-[3-(pyrrolidin-1-yl)propyl]propanamide; N-{[ethyl(2-hydroxyethyl)amino]methyl}-3-(4-methoxyphenoxy)-N-methylpropanamide; 2-(3-fluoro-4-hydroxyphenoxy)-N-[2-(1H-pyrrol-1-yl)ethyl]acetamide; N-[2-(diethylamino)ethyl]-N-ethyl-2-(4-methoxyphenoxy)acetamide; 2-[4-({[3-(1H-pyrrol-1-yl)propyl]carbamoyl}methoxy)phenyl] acetic acid; 2-(4-methoxyphenoxy)-N-methyl-N-[2-(piperidin-1-yl)ethyl] acetamide; 2-(4-methoxyphenoxy)-N-[2-(piperidin-1-yl)ethyl] acetamide; 2-(4-methoxyphenoxy)-N-[2-(morpholin-4-yl)ethyl]acetamide; and 2-(3-fluoro-4-methoxyphenoxy)-N-[2-(morpholin-4-yl)ethyl] acetamide,
or a pharmaceutically acceptable salt thereof or stereoisomer of said compound or salt.

30. A method of inhibiting the biological activity of High Mobility Group Protein B1 comprising administering to a mammal an effective amount of a compound of Formula I below 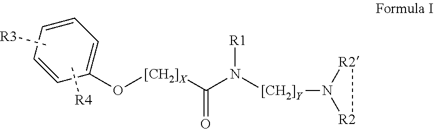
wherein
X=1-3;
Y=1-3;
R1=is independently selected from methyl, ethyl, propyl, butyl, haloalkyl, or hydrogen;
R2=is independently selected from methyl, ethyl, propyl, butyl, alkoxy, haloalkyl, or hydrogen;
R2′=is independently selected from methyl, ethyl, propyl, butyl, alkoxy, haloalkyl, or hydrogen;
R2 & R2′ may be fused into a heterocyclic ring with 5 or 6 members;
R3=is independently selected from methyl, ethyl, hydroxyl, alkoxy, carboxylic acid, haloalkyl, or hydrogen; and
R4=is independently selected from hydrogen, hydroxyl or halogen,
or a pharmaceutically acceptable salt, addition compound or pro-drug thereof.

31. The method of claim 30, wherein the compound moderates the interaction of High Mobility Group Protein B1 with at least one ligand of High Mobility Group Protein B1 and/or wherein the compound reduces the genetic expression of at least one ligand of High Mobility Group Protein B1.

32. The method of claim 30, wherein the compound moderates the biological activity of at least one protein in the myeloid differentiation primary response protein dependent intracellular signaling pathway and/or the compound decreases the gene expression of at least one protein in the myeloid differentiation primary response protein dependent intracellular signaling pathway.

33. The method of claim 30, wherein the compound according to Formula I is a racemic mixture or is a specific stereoisomer.

34. A method of treatment comprising administering to a mammal in need thereof, including a human, a therapeutically effective amount of a compound of Formula 1 below 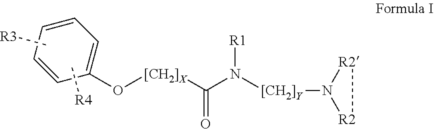
wherein
X=1-3;
Y=1-3;
R1=is independently selected from methyl, ethyl, propyl, butyl, haloalkyl, or hydrogen;
R2=is independently selected from methyl, ethyl, propyl, butyl, alkoxy, haloalkyl, or hydrogen;
R2′=is independently selected from methyl, ethyl, propyl, butyl, alkoxy, haloalkyl, or hydrogen;
R2 & R2′ may be fused into a heterocyclic ring with 5 or 6 members;
R3=is independently selected from methyl, ethyl, hydroxyl, alkoxy, carboxylic acid, haloalkyl, or hydrogen; and
R4=is independently selected from hydrogen, hydroxyl or halogen,
or a pharmaceutically acceptable salt, addition compound or pro-drug thereof for treatment of a disease or condition selected from the group consisting of autoimmune diseases; inflammatory diseases; autoinflammatory conditions; pain conditions; respiratory; airway and pulmonary conditions; gastrointestinal disorders; allergic diseases; infection-based diseases; trauma and tissue injury-based conditions; fibrotic diseases; ophthalmic/ocular diseases; joint, muscle and bone disorders; skin/dermatological diseases; renal diseases; genetic diseases; hematopoietic diseases; liver diseases; oral diseases; metabolic diseases, including diabetes (e.g. Type II) and complications thereof; proliferative diseases; cardiovascular conditions; vascular conditions; neuro-inflammatory conditions; neurodegenerative conditions; cancer; sepsis; pulmonary inflammation and injury; and pulmonary hypertension.

35. The method according to claim 34, wherein the disease or condition is selected from the group consisting of autoimmune diseases; respiratory; airway and pulmonary conditions; gastrointestinal disorders; allergic diseases; infection-based diseases; trauma and tissue injury-based conditions; fibrotic diseases; ophthalmic/ocular diseases; joint, muscle and bone disorders; skin/dermatological diseases; renal diseases; genetic diseases; hematopoietic diseases; liver diseases; oral diseases; metabolic diseases, including diabetes (e.g. Type II) and complications thereof; proliferative diseases; cardiovascular conditions; vascular conditions; neuro-inflammatory conditions; neurodegenerative conditions; cancer; sepsis; pulmonary inflammation and injury; and pulmonary hypertension.

36. The method of claim 34, further comprising administering at least one other compound selected from the group consisting of: non-steroidal anti-inflammatory drugs, immunomodulatory and/or anti-inflammatory agents, antimalarials, antibiotics, Anti-TNFα agents, Anti-CD20 agents, Antidiarrheals, Bile acid binding agents, laxatives, T lymphocyte activation inhibitors, Anti-IL1 treatments, Glucocorticoid receptor modulators, Aminosalicyic acid derivatives including but not limited to: sulfasalazine and mesalazine, Anti-α4 integrin agents, adrenergic agonist agents, anticholinergic agents, inhaled long acting beta-agonists, long acting muscarinic antagonists, long acting corticosteroids, leukotriene pathway modulators, and H1 receptor antagonists.

37. The method of claim 34 wherein the compound according to Formula 1 is a racemic mixture or a specific stereoisomer.

38. A method of decreasing the gene expression of NF-kB, TLR2, CCL2, VCAM and/or combinations thereof and/or decreasing the production of Interleukin 17, Interleukin 6 TNF alpha and/or combinations thereof by human cells in the presence of extracellular HMGB1 by administering an effective amount of a compound of Formula I below 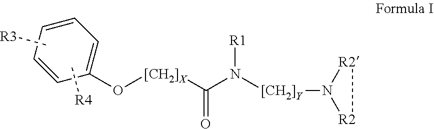
wherein
X=1-3;
Y=1-3;
R1=is independently selected from methyl, ethyl, propyl, butyl, haloalkyl, or hydrogen;
R2=is independently selected from methyl, ethyl, propyl, butyl, alkoxy, haloalkyl, or hydrogen;
R2′=is independently selected from methyl, ethyl, propyl, butyl, alkoxy, haloalkyl, or hydrogen;
R2 & R2′ may be fused into a heterocyclic ring with 5 or 6 members;
R3=is independently selected from methyl, ethyl, hydroxyl, alkoxy, carboxylic acid, haloalkyl, or hydrogen; and
R4=is independently selected from hydrogen, hydroxyl or halogen,
or a pharmaceutically acceptable salt, addition compound or pro-drug thereof.

39. The method of claim 38 wherein the compound according to Formula 1 is a racemic mixture or a specific stereoisomer.
